(19) United States
(12) Patent Application Publication
(10) Pub. No.: US 2016/0151285 A1
(43) Pub. Date: Jun. 2, 2016

(54) METHOD TO PRODUCE SLOW RELEASE DELIVERY CARRIER LIPID NANOPARTICLES OF DIFFERENT LOG P VALUE

(71) Applicant: University of Malaya, Kuala Lumpur (MY)

(72) Inventors: Misni Bin Misran, Kuala Lumpur (MY); Shu Xian Tiew, Dengkil (MY); Juin Onn Woo, Kuala Lumpur (MY)

(21) Appl. No.: 14/952,291

(22) Filed: Nov. 25, 2015

(30) Foreign Application Priority Data
Nov. 28, 2014 (MY) 2014703562

(51) Int. Cl. A61K 9/14; A61K 31/4706

(52) U.S. Cl. CPC A61K 9/145; A61K 31/4706

(57) ABSTRACT

The present invention disclosed a method to produce a slow release lipid nanoparticles comprising steps of determining a log P value of an active ingredient; mixing fatty acid, non-ionic surfactant and the active ingredient to form a mixture; melting the mixture; homogenizing the mixture to form a nanoemulsion; sonicating the homogenized nanoemulsion; and pouring the nanoemulsion into cold water to form the liquid nanoparticles. The lipid nanoparticles with active ingredient and the selected log P value has a prolong release property. In specific embodiment, the active ingredients are the chemical compound with the log P value selected from the range 0 to 4.0.

### CROSS-REFERENCE TO RELATED APPLICATIONS

This application claims the benefit of and priority to Malaysian Patent Application No. PI 2014703562 filed on Nov. 28, 2014, which application is incorporated herein by reference in its entirety.

### BACKGROUND OF THE INVENTION

The present invention relates to nanostructure lipid carriers for cosmetic and pharmaceutical product application. More specifically, the present invention relates to a method of producing lipid nanoparticles which are able to provide slow release of active ingredients to the targeted site.

Solid lipid nanoparticles (SLNs) have attracted increased interest as its nanometer-sized drug delivery systems combine the advantages of polymeric nanoparticles, emulsion and liposomes (Paliwal et al., 2009; Subedi et al., 2009). SLNs are essentially consists of a biocompatible lipid matrix with surfactant at the outer shell, which are in solid state at room temperature (Kamboj et al., 2010). Due to their unique structure and properties, such as good biocompatibility, protection for the incorporated compound against degradation and sustained release of drugs, SLNs have been used widely as carrier for the drug and active ingredients delivery system for pharmaceutical, cosmetic and dermatological purposes (Chourasia and Jain, 2009; Zhang et al., 2012). However, the drawbacks of SLN are inherent as a result of the perfect crystalline state of the nanoparticles which caused by unexpected polymorphic transitions with lipid transforming to more perfect β-modification (Freitas and Miller, 1999), creating potential problems like limited drug entrapment and drug expulsion.

Nanostructured lipid carriers (NLC), a new generation of lipid nanoparticles were developed to overcome the limitations (Pardeike et al., 2009). NLC or lipid nanoparticles (hereinafter named as “lipid nanoparticles”) are sub-micron sized lipid particles that contained a blend of solid lipid and liquid oil. Incorporation of liquid oil into lipid nanoparticles perturbs the perfect crystal arrangement of the solid lipid matrix with their differences in structure, inducing lots of imperfections to accommodate drug in molecular form and hence help to improve the loading capacity of the active ingredients (Muller et al. 2002; Souto and Muller, 2006).

Different types of raw materials were used to prepare lipid nanoparticles such as triacylglycerides, phospholipids and waxes (Wang et al., 2011, Kheradmandnia et al., 2010, Jenning et al., 2000, Tsai et al., 2012 and China Patent No. CN101890170). The major drawback of these prior arts is the cost of the raw material used for the preparation of lipid nanoparticles is expensive. It would be advantageous to have a cheaper compound to use as the raw materials for the preparation of lipid nanoparticles. It would also be advantageous to develop a simple method for producing lipid nanoparticles from the cheaper raw materials. Besides, there is no available lipid nanoparticles that are able to provide slow release of active ingredients to the targeted site.

The release of drug such as controlled release or sustained release are important in drug delivery system in order to achieve prolonged therapeutic effects by continuously releasing medication over an extended period of time after administration of a single dose (Chuga Isha et al., 2012). Log P is the octanol-water partition coefficient, which is a good indication of the lipid solubility of the drugs or active ingredients; with a higher log P value describes higher solubility in the lipid environment (Hansch & Clayton, 1973).

In view of the foregoing, the present invention has developed a method to produce a slow release lipid nanoparticle delivery carrier for use in cosmetic or pharmaceutical application. In this method, the lipid nanoparticle is fabricated using cheaper compound—fatty acid as the raw material of the lipid nanoparticles. The lipid nanoparticles according to the present invention consist of fatty acid, non-ionic surfactant and active ingredients which the active ingredients are chemical compound with selected log P value. In addition, the fabricated lipid nanoparticles delivery carrier produced according to the method of the present invention has a prolong release property which is suitable for cosmetic or pharmaceutical application.

### BRIEF SUMMARY OF SOME EXAMPLE EMBODIMENTS

This Summary is provided to introduce a selection of concepts in a simplified form that are further described below in the Detailed Description. This Summary is not intended to identify key features or essential characteristics of the claimed subject matter, nor is it intended to be used as an aid in determining the scope of the claimed subject matter.

An object of the present invention is to provide a method of producing a slow release lipid nanoparticles delivery carrier by using saturated or unsaturated fatty acid with 16 to 24 carbon atoms as an encapsulated raw material. The method of producing a slow release lipid nanoparticles delivery carrier disclosed according to the present invention comprising steps of: determining the log P value of an active ingredient; mixing fatty acid, non-ionic surfactant and the active ingredient to form a mixture; melting the mixture at the temperature of 75° C. to 90° C.; waiting until all ingredients are melted, homogenizing the mixture for 2 to 15 minutes at 13,000 to 20,000 rpm to form a nanoemulsion; sonicating the homogenized nanoemulsion for 1 to 2 minutes to reduce the particle size and polydispersity index; and pouring the nanoemulsion into a cold water with temperature equal to or below 5° C. to form the liquid nanoparticles. In a preferred embodiment, the active ingredients are selected from the chemical compound with the log P value selected from the range of 0 to 4.0.

Another object of the present invention is to provide a slow release lipid nanoparticle delivery carrier for cosmetic and pharmaceutical application consists of saturated or unsaturated fatty acid with 16 to 24 carbon atoms as an encapsulated raw material to encapsulate active ingredients and stabilized by non-ionic surfactant. The lipid nanoparticle delivery carrier according to the present invention has a prolong release property.

These and other objects and features of the present invention will become more fully apparent from the following description and appended claims, or may be learned by the practice of the invention as set forth hereinafter.

### BRIEF DESCRIPTION OF THE DRAWINGS

To further clarify various aspects of some example embodiments of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1 is a flowchart illustrating the operational steps of the disclosed method of producing lipid nanoparticles to encapsulate the compound of high log P value;

FIG. 2 shows the in vitro release of four different samples over a period of 24 hours for chloroquine diphosphate; ▪ in solution, [symbol] in cream, [symbol] in NLC and [symbol] in NLC blended cream;

FIG. 3 shows the in vitro release of four different samples over a period of 24 hours for acridine orange; ▪ in solution, [symbol] in cream, [symbol] in NLC and [symbol] in NLC blended cream;

FIG. 4 shows the in vitro release of four different samples over a period of 24 hours for arsenazo III; ▪ in solution, [symbol] in cream, [symbol] in NLC and [symbol] in NLC blended cream;

FIG. 5 shows the cumulative release of four different samples at 37° C. for chloroquine diphosphate; ▪ in solution, [symbol] in cream, [symbol] in NLC and [symbol] in NLC blended cream;

FIG. 6 shows the cumulative release of four different samples at 37° C. for acridine orange; ▪ in solution, [symbol] in cream, [symbol] in NLC and [symbol] in NLC blended cream; and

FIG. 7 shows the cumulative release of four different samples at 37° C. for arsenazo III; ▪ in solution, [symbol] in cream, [symbol] in NLC and [symbol] in NLC blended cream.

### DETAILED DESCRIPTION OF SOME EXAMPLE EMBODIMENTS

Reference will now be made to the figures wherein like structures will be provided with like reference designations. It is understood that the figures are diagrammatic and schematic representations of some embodiments of the invention, and are not limiting of the present invention, nor are they necessarily drawn to scale.

In general, the present invention represents a method of producing a slow release lipid nanoparticles delivery carrier. The lipid nanoparticles delivery carrier produced according to the present invention has a prolong release property and with a diameter in the range of 50 nm to 500 nm.

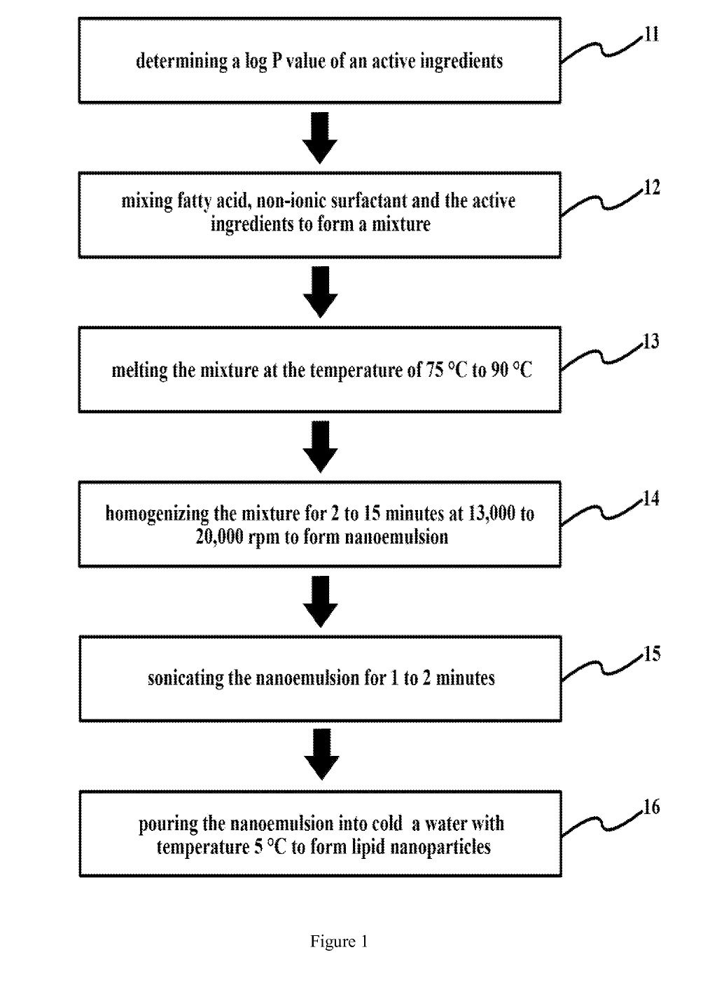

FIG. 1 is an example of a representative flowchart of a method of the present invention. This flowchart is merely an illustration and should not limit the scope of the claims herein. One of ordinary skilled in the art will recognize other variations, modifications and alternatives. Referring to FIG. 1, the method to produce the slow release lipid nanoparticles according to the present invention comprising steps of: determining a log P value of an active ingredient, indicated by reference numeral (11); mixing fatty acid, non-ionic surfactant and the active ingredient to form a mixture, indicated by reference numeral (12); melting the mixture at the temperature 75° C. to 90° C., indicated by reference numeral (13); homogenizing the mixture for 2 to 15 minutes 13,000 to 20,000 rpm to form a nanoemulsion, indicated by reference numeral (14); sonicating the homogenized nanoemulsion for 1 to 2 minutes to reduce the particle size and polydispersity index, indicated by reference numeral (15); and pouring the nanoemulsion into a cold water with temperature equal to or below 5° C. to form liquid nanoparticles, indicated by reference numeral (16).

Operational step (11) is the beginning step, in this step, the log P value of the active ingredient is determined. In one embodiment, the active ingredient intended to encapsulate into a lipid nanoparticles must consists of log P value selected from the range of 0 to 4.0.

Next, at operational step (12), the fatty acid, non-ionic surfactant and the active ingredient are mixed to form a mixture. In another embodiment, the fatty acid used as the raw material of the lipid nanoparticles is selected from the group consisting of saturated or unsaturated fatty acid with the number of carbon atoms from 16 to 24. In an exemplary embodiment, a wide variety of materials can be utilized as the lipid nanoparticles raw material, including but not limited to, stearic acid, oleic acid, palmitic acid, arachidic acid, behenic acid, lignoceric acid, palmitoleic acid, sapienic acid, elaidic acid, vaccenic acid, linoleic acid, arachidonic acid, vaccenic acid, erucic acid or any other saturated or unsaturated fatty acid with the number of carbon atoms from 16 to 24.

In yet another embodiment, the non-ionic surfactant used in operational step (12) is selected from the group consisting of polysorbates (Tween™), sorbitan oleate (Span™), sucrose fatty acid esters, ceteareth, ceteth, polyoxyethers of lauryl alcohol (laureth), poloxamer or the like. Surfactants are usually organic compounds that are amphiphilic, meaning they contain both hydrophobic groups (their tails) and hydrophilic groups (their heads). Therefore, a surfactant contains both a water insoluble (or oil soluble) component and a water soluble component. Most commonly, surfactants are classified according to polar or hydrophilic head group. A non-ionic surfactant has no charge groups in its head. The head of an ionic surfactant carries a net charge. If the charge is negative, the surfactant is more specifically called anionic; if the charge is positive, it is called cationic. If a surfactant contains a head with two oppositely charged groups, it is termed zwitterionic. In the present invention, the function of the non-ionic surfactants is to stabilize the lipid nanoparticles formed by the fatty acid.

Still referring to FIG. 1, at operational step (13), the mixture of fatty acid, non-ionic surfactant and the active ingredients are melted at the temperature of 75° C. to 90° C. In still another embodiment, the mixture of the fatty acid, active ingredients and non-ionic surfactant containing solid and liquid phase substances. Thus, in order to homogenize the mixture, the mixture must be melted to change all the substances into liquid phase. The temperature applies to melt the mixture of the fatty acid, active ingredient and non-ionic surfactant is preferably arranged at 75° C. to 90° C. The melting point for different substances is different; however, at the temperature of 75° C., most of the substances used to produce lipid nanoparticles will start to melt at this temperature. It is not advisable to heat the mixture at more than 90° C. as higher temperature will cause damage or decomposition to the active ingredients. This will decrease the pharmaceutical effect of the lipid nanoparticles.

After all the ingredients are melted, at the operational step (14), the mixture is homogenized for 2 to 15 minutes at 13,000 to 20,000 rpm to form a nanoemulsion. Next, at the operational step (15), the homogenized nanoemulsion is sonicated for 1 to 2 minutes to reduce the particle size and polydispersity index. At operational step (16), the nanoemulsion is poured into cold water with temperature equal to or below 5° C. to form liquid nanoparticles.

In yet another embodiment, the sizes of lipid nanoparticles that are obtained by using the operational steps (11) to (16) according to the present invention are in the range of 50 nm-500 nm. Operation step (14) is an optional step and it is much more depending on how small the lipid nanoparticles that are intended to achieve. Besides, the desired particle size of lipid nanoparticles can be obtained by controlling the homogenization time or speed, applying sonification and varying surfactant concentration.

To further illustrate the present invention in greater details and not by way of limitation, the following examples will be given. To provide a better understanding and for comparison purposes, in the following examples, besides using a pure saturated fatty acid (stearic acid) to produce lipid nanoparticles, a mixture of saturated fatty acid (stearic acid) and monounsaturated fatty acid (oleic acids) have also been used to produce lipid nanoparticles.

### Example 1

### Preparation of Lipid Nanoparticles

Stearic acid was purchased from Sigma-Aldrich (St. Louis, USA). Oleic acid was obtained from Fluka (Buchs, Switzerland). Tween 60 (Polyoxyethylene sorbitan monostearate) was purchased from Lasem Asia (Kuala Lumpur, Malaysia). Compounds used such as chloroquine diphosphate salt, acridine orange and arsenazo III were purchased from Sigma-Aldrich (St. Louis, USA), Tokyo Chemical Industry, TCI (Japan) and Merck (Germany) respectively. Salcare SC91 was obtained from BASF (Ludwigshafen, Germany). Deionized water with resistivity of 18.2 OS cm-¹ was used to prepare all solutions and samples (Barnstead NANO Pure® Diamond™ Nanopure water system).

Lipid nanoparticles were prepared by using a melt-emulsification technique. 400 mg stearic acid was mixed with oleic acid at 30 wt % OA ratio to be melted at 80° C. and then mixed with Tween 60 solution. The mixture was homogenized for 10 minutes, then, further sonicated for 2 minutes to form nanoemulsion. The nanoemulsion was poured into 20 ml of cold water (2° C.) to form lipid nanoparticles.

For better understanding and for comparison purpose, compounds of different log P have been encapsulated in the lipid nanoparticles. Three compounds of different log P, chloroquine diphosphate salt (log P: 3.93), acridine orange (log P: 3.72), arsenazo III (log P: 0.49) were used. Chloroquine diphosphate has been widely used as a potent antimalarial and amebicidal drug, as well as treatment drugs (Jiang et al., 2010). Acridine orange is the nitrogen-containing analogues of anthracene which used as fluorescence dyes in molecular biology, biochemistry, toxicology and supramolecular chemistry (Shaikh et al., 2008; Agrayat et al., 2008). Arsenazo III which is a water-soluble compound is a metallochromic indicator of calcium, uranium, cadmium and so on (Brown and Rydqvist, 1981).

Chloroquine diphosphate salt, acridine orange and arsenazo III loaded stearic acid-oleic acid nanoparticles (SON) dispersions were prepared exactly in the same manner as stated above, but, only adding 10 mg of different compounds separately into lipid phases above to form NLC-1, NLC-2 and NLC-3 respectively. Chloroquine diphosphate salt, acridine orange and arsenazo III loaded SON dispersions were further incorporated into Salcare SC91 base cream obtained from BASF, a raw material that consisted of 8% anionic acrylic copolymer dispersed in a 5-8% of medical grade white oil by vortex technique, where thee samples are denoted as NLC-C1, NLC-C2 and NLC-C3 respectively. The blank chloroquine diphosphate salt, acridine orange and arsenazo III solution and the respective blank compounds dissolving only base cream were prepared for comparison as well with denotion as A1, A2, A3 respectively for solution and C1, C2, C3 respectively for cream. All the samples (NLC-1, NLC-2, NLC-3; NLC-C1, NLC-C2, NLC-C3; A1, A2, A3 and C1, C2, C3) were stored at 5° C. after preparation.

### Example 2

### Characterization of Lipid Nanoparticles

Particle Size and Zeta Potential of Lipid Nanoparticles

The average particle size and zeta potential of compounds loaded lipid nanoparticles were determined by using Zetasizer ZS (Malvin Instrucments, UK). 500 μl of lipid nanoparticles was diluted to 25 ml with deionised water and was equilibrated to room temperature for 10 minutes. Particle size and zeta potential measurements were performed at 25° C.

Encapsulation Efficiency (EE) of Lipid Nanoparticles

Compounds loaded SON dispersion was centrifuged in an upper chamber of centrifugal filter tubes with 50,000 Da molecular weight cut-off (Vivaspin 6, Sartorius Stedim Biotech, Germany) for 30 min at 8000 rpm. The amount of free compound collected at the bottom of the centrifugal filter tubes was diluted and determined spectrophotometrically with Cary 50 UV-Vis Spectrometer (Agilent Technologies, USA). A standard calibration curve was constructed to determine the concentration of the compound using equation below:

$$EE = ( \frac{WT - WF}{WT} ) \times 100 \%$$

EE: Encapsulation efficiency of NLC

WT: The weight of salicylic acid added during preparation

WF: The weight of unloaded salicylic acid collected at the bottom of the tube

### Example 3

### In Vitro Release Study

The in vitro drug release of the four different samples which are compound dissolving solution only (A1, A2, A3), compounds encapsulated lipid nanoparticles (NLC-1, NLC-2, NLC-3), compounds dissolving base cream (C1, C2, C3) and compounds encapsulated lipid nanoparticles blended cream (NLC-C1, NLC-C2 and NLC-C3) for each compound were evaluated by using Automated Franz Diffusion Cell System (Microette Autosampling System, Hanson Research Co., USA).

Four different samples were run in 6 diffusate chamber volume of 4 ml 10 mm PBS solution at pH7.4, with 0.6362 cm² effective diffusion area, under 400 rpm stirring and circulation of water bath at 37±1° C. throughout the experiments. Approximately 500 mg of each sample was loaded on the regenerated cellulose membrane surface (5000 Da molecular weight cut-off) in the retentate compartment, which was pre-soaked overnight, to be evaluated for 24 hours. At predetermined intervals, samples were withdrawn to be determined spectrophotometrically for the drug content and replaced with fresh receiving medium. The calculations are done based on average of six measurements from six different retentate compartments.

Results and Discussions

Particle Size and Zeta Potential of Lipid Nanoparticles

Particle size and zeta potential of lipid nanoparticles were showed in Table 1. The particle size of NLC-1 showed the biggest particle size compared to NLC-2 & NLC-3 with its highest log P value. It may be due to high solubility of chloroquine diphosphate salt in water (100 mg/ml) (Kasim et al., 2004) which causes an incompatible mixing between the drug and crystalline layers of lipid matrix of OA and stearic acid. Same reason is suited to explain the bigger size of NLC-3 compared to NLC-2 which possesses smaller lipophilicity with tendency to put in polar aqueous phase rather than a lipophilic lipid phase. There is no significant difference in polydispersity index among all three NLCs of different log P.

TABLE 1
The particle size, polydispersity index, zeta potential and EE of
freshly prepared lipid nanoparticles at 25° C.

| | | Particle | | Zeta potential | |
|---|---|---|---|---|---|
| Samples | Log P | size (nm) | PDI | (mV) | EE (%) |
| NLC-1 | 3.93 | 414 ± 5 | 0.41 ± 0.03 | −29.6 ± 0.3 | 79.57 |
| NLC-2 | 3.72 | 241 ± 1 | 0.40 ± 0.01 | −11.1 ± 0.1 | 89.26 |
| NLC-3 | 0.49 | 356 ± 2 | 0.46 ± 0.03 | −40.8 ± 0.2 | 93.32 |

Zeta potential is the electrical potential across the electrical double layer which surrounding the particles (Elazhary and Soliman, 2009). It is one of the parameters to predict the stability of the colloidal dispersion system (Muller et al., 2001). In general, particles could be dispersed stably when absolute value of zeta potential is above 30 mV due to the electric repulsion between particles (Muller et al., 2000). Referring to table 1, NLC-1 was found near to −30 mV whereas NLC-2 showed zeta potential lower than −40 mV, indicating good stability of storage. On the contrary, zeta potential of NLC-2 is higher than −30 mV as acridine orange exist in their protonated form under physiologic conditions (pH=7.4) (Lagutschenkov and Dopfer, 2011), resulting the negative charge at the interface of NLC was being neutralized by the positive charge of acridine orange and showed smaller negative value of zeta potential.

Referring to table 1, EE of NLCs were showing decreasing tendency with increasing log P value. This may be explained by incompatible mixing between the drug and lipid core of NLC, limiting the loading of the compounds into the lipid matrix as lipophilicity is increasing.

In Vitro Release Study of Lipid Nanoparticles

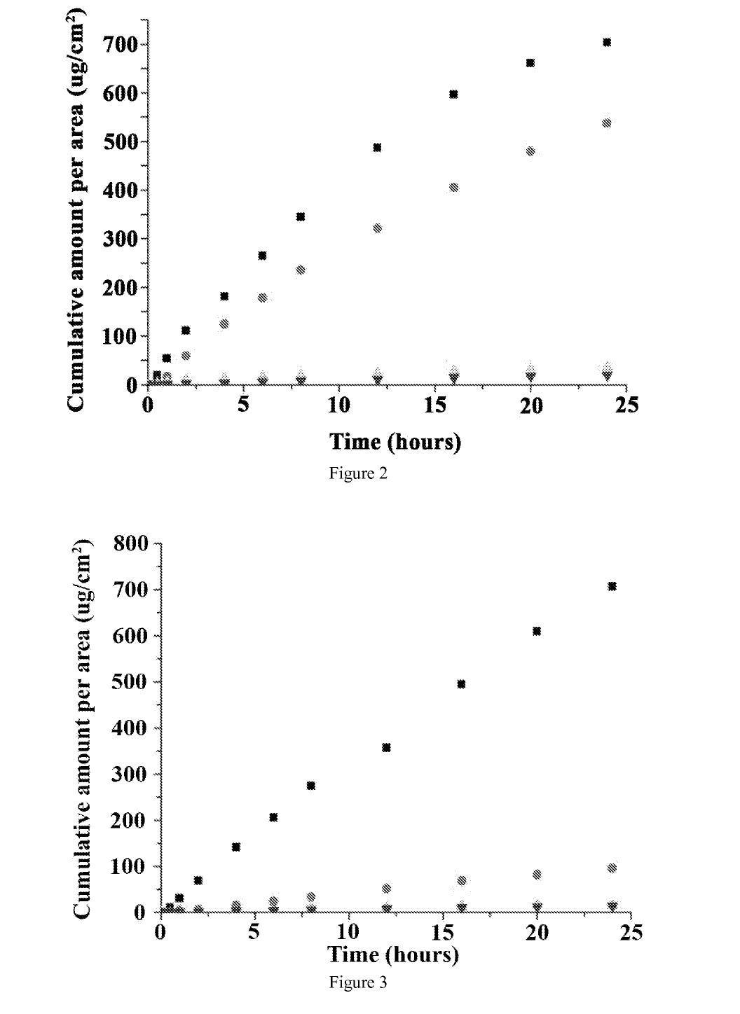

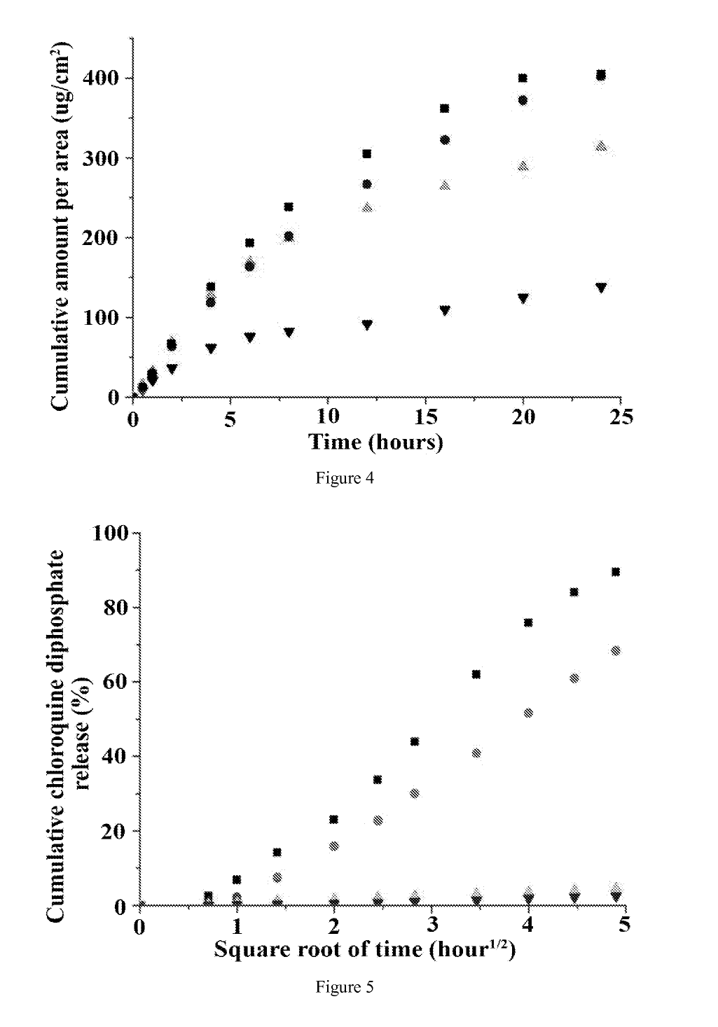

Four different samples as described above were evaluated using Static Franz Diffusion Cell method and the cumulative compound release from all samples was plotted against time. Referring to FIGS. 2, 3 and 4, it can be noticed that the higher the log P of the compound, the more the compound amount diffuse throughout the membrane, proved by higher amount released of A1, following with A2 and A3. The reason of this phenomenon is caused by thick lipid layers of stratum corneum of skin epidermis that drugs should pass through stratum corneum for permeation. Lipophilic molecule will easily partition in stratum corneum but will leave it with difficulty, whereas a hydrophilic molecule will suffer poor penetration (Pouillot et al, 2008; Desai et al. 2010).

In FIGS. 2, 3 and 4, all the compounds showed gradual release within 24 hours, however, the compounds are releasing slower with the NLC carrier regardless of log P of the compounds. The encapsulated drug in particles diffuses to the surface of the inner phase and undergoes partitioning between the lipid and aqueous phases (Venkateswarlu and Manjunath, 2004). NLC-1 which has the highest log P showed the higher amount of drug diffused compared to NLC-2 & NLC-3.

NLC-C1, NLC-C2 and NLC-C3 demonstrated slower release trend rather than NLC-1, NLC-2 and NLC-3 since the compounds are retained in the NLC particle dispersion and in the emulsion. NLC-1, NLC-2, NLC-3, NLC-C1, NLC-C2 and NLC-C3 are released at a slower rate as compared to the samples without SON (A1, A2, A3, C1, C2, C3). The presence of controlled release behaviour for samples containing SON revealed that the compounds were successfully incorporated in the solid matrix of SON and slowly released from the particles.

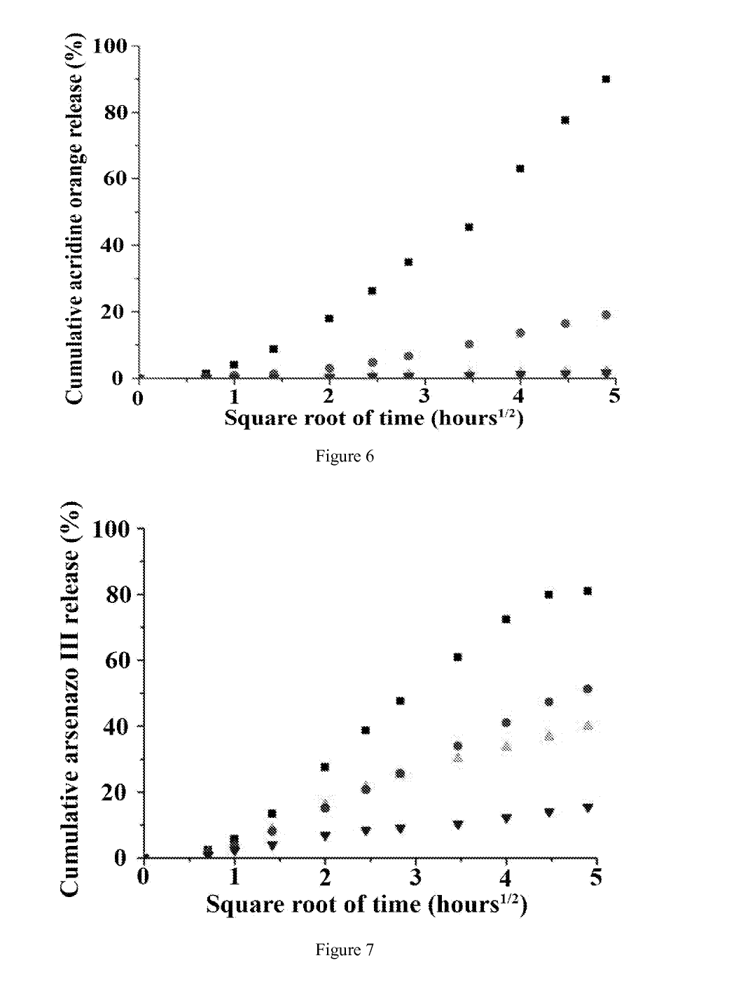

The in vitro releases of the four different samples were curve-fitted to Higuchi model in order to understand their release kinetics since this model is used to describe the diffusion of drug from homogenous and granular matrix system (Vivek et al., 2007). The release profiles of the four samples for every compounds showed the best fit into Higuchi model (R²>0.93). Linear fits for all samples were obtained (FIGS. 5, 6 and 7), denoting that the release of compounds from the samples was diffusion controlled process (Vivek et al. 2007). The slopes obtained from the plotting of Higuchi model as shown in table 2 represent the release rate of compounds. The release rate among the four samples were A1>C1>NLC-1>NLC-C1 for chlorquine diphosphate, A2>C2>NLC-2>NLC-C2 for acridine orange, A3>C3>NLC-3>NLC-C3 for arsenazo III, respectively. The release rate of every compound (A1, A2, A3) was reduced when formulated into cream (C1, C2, C3) revealing this retentional effect by the emulsion. The release rate of C1, C2, C3 is further decreased to about 40, 12, 4 times, respectively when the respective compounds loaded NLC was incorporated into cream (NLC-C1, NLC-C2, NLC-C3) and this indicates that the NLC prepared in the present invention has a slow release property. This slow release property of NLC could be due to the release of compounds from the solid matrix of NLC particle involved two pathways. First pathway involved the slow release of the compound in the retentate chamber (k₁) and followed by second pathway of diffusion through the membrane at the faster rate (k₂>k₁). The reason of slow release of compounds at the first pathway may be due to the hydrophobic solid matrix of NLC retaining the release of compounds to the aqueous phase and thus, results and prolonged release rate.

TABLE 2
Compound release of four different samples
according to the Higuchi Model

| Compounds | | |
|---|---|---|
| Chloroquine | Acridine | Arsenazo |
| diphosphate | orange | III |

| Samples | Slope | R² | Slope | R² | Slope | R² |
|---|---|---|---|---|---|---|
| In solution | 20.54 | 0.97 | 19.15 | 0.93 | 19.25 | 0.98 |
| In cream | 16.69 | 0.99 | 4.12 | 0.94 | 11.48 | 0.99 |
| In NLC | 0.91 | 0.99 | 0.43 | 0.99 | 8.93 | 0.98 |

### CONCLUSION

In summary, the present invention has demonstrated a method to produce the slow release lipid nanoparticles by melt-emulsification technique. The particles size of compound loaded NLC will increase, but, oppose in encapsulation efficiency, with the increasing of log P value due to incompatible mixing between the compound and crystalline layer of lipid matrix of OA and stearic acid. Higher Log P of compounds showed higher diffusate across the lipid layers of stratum corneum of skin epidermis, both in NLC and emulsions. The compounds loaded NLC prepared in the present invention has a prolong release property which may be due to the incorporation of compounds in solid matrix of NLC. The incorporation of NLC into cream formulation further reduced the release rate over a period of 24 hours revealing that the emulsion also slowed down the release of compound. The present invention suggests that drug loaded NLC enriched cream could be a promising delivery system for the enhancement of the therapeutic efficacy in the cosmetic and pharmaceutical application.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A method of producing a slow release lipid nanoparticles delivery carrier for cosmetic or pharmaceutical application comprising the steps of:
determining a log P value of an active ingredient;
mixing fatty acid, non-ionic surfactant and the active ingredient to form a mixture;
melting the mixture at the temperature of 75° C. to 90° C.;
waiting until all the ingredients are melted, homogenizing the mixture for 2 to 15 minutes at 13,000 to 20,000 rpm to form a nanoemulsion;
sonicating the homogenized nanoemulsion for 1 to 2 minutes to reduce the particle size and polydispersity index; and
pouring the nanoemulsion into cold water with temperature equal to or below 5° C. to form the lipid nanoparticles.

2. The method according to claim 1, wherein the active ingredients are the chemical compound with the log P value selected from the range of 0 to 4.0.

3. The method according to claim 1, wherein the lipid nanoparticles has a prolong release property and with a diameter in the range of 50 nm to 500 nm.

4. The method according to claim 1, wherein the fatty acid is selected from the group consisting of saturated or unsaturated fatty acid with the number of carbon atoms from 16 to 24.

5. The method according to claim 4, wherein the saturated or unsaturated fatty acid with the number of carbon atoms from 16 to 24 is selected from the group consisting of:
lauric acid;
myristic acid;
palmitic acid;
stearic acid;
arachidic acid;
behenic acid;
lignoceric acid;
myristoleic acid;
palmitoleic acid;
sapienic acid;
oleic acid;
elaidic acid;
vaccenic acid;
linoleic acid;
arachidonic acid;
vaccenic acid; and
erucic acid.

6. The method according to claim 1, wherein the non-ionic surfactant is selected from the group consisting of:
polysorbates, Tween;
sorbitan oleate, Span;
sucrose fatty acid esters;
ceteareth;
ceteth;
polyoxyethers of lauryl alcohol, laureth; and
poloxamer.

7. A slow release lipid nanoparticles delivery carrier consists of a prolong release property produced by the method of claim 1.
